(19) United States
(12) Patent Application Publication
(10) Pub. No.: US 2002/0138147 A1
(43) Pub. Date: Sep. 26, 2002

(54) Apparatus for fusing adjacent bone structures

(75) Inventor: Herb Cohen, Shelton, CT (US)

Correspondence Address: Lawrence Cruz, Esq., United States Surgical, a division of, TYCO HEALTHCARE GROUP LP, 150 Glover Avenue, Norwalk, CT, 06856, US

(73) Assignee: Surgical Dynamics, Inc.

(21) Appl. No.: 09/815,066

(22) Filed: Mar. 22, 2001

(51) Int. Cl. A61F 2/44

(52) U.S. Cl. 623/17.16

(57) ABSTRACT

An apparatus and method for spinal fusion includes an implant having a plurality of side walls connected to each other to define an enclosed structure. The implant is advantageously dimensioned to facilitate insertion and retention between adjacent vertebrae, and enhance fusion with adjacent vertebral bones.

### BACKGROUND

1. Technical Field

The present disclosure generally relates to a surgical apparatus for fusing adjacent bone structures, and, more particularly, to an apparatus and associated method for fusing adjacent vertebrae.

2. Background of the Related Art

The fusion of adjacent bone structures is commonly performed to provide for long-term replacement to compensate for degenerative or deteriorated disorders in bone. For example, an intervertebral disc, which is a ligamentous cushion disposed between adjacent vertebrae, may undergo deterioration as a result of injury, disease, tumor or other disorders. The disk shrinks or flattens leading to mechanical instability and painful disc translocations.

Conventional procedure for disc surgery include partial or total excision of the injured disc portion, e.g., discectomy, and replacement of the excised disc with biologically acceptable plugs or bone wedges. The plugs are driven between adjacent vertebrae to maintain normal intervertebral spacing and to achieve, over a period of time, bony fusion with the plug and opposed vertebrae. More recently, emphasis has been placed on fusing bone structures (i.e., adjoining vertebrae) with prosthetic cage implants. One fusion cage implant is disclosed in commonly assigned U.S. Pat. No. 5,026,373 to Ray et al., the contents of which are incorporated herein by reference. The Ray '373 fusion cage includes a cylindrical cage body having a thread formed as part of its external surface and apertures extending through its wall which communicate with an internal cavity of the cage body. The fusion cage is inserted within a tapped bore or channel formed in the intervertebral space thereby stabilizing the vertebrae and maintaining a pre-defined intervertebral space. Preferably, a pair of fusion cages are implanted within the intervertebral space. The adjacent vertebral bone structures communicate through the apertures and with bone growth inducing substances which are within the internal cavity to unite and eventually form a solid fusion of the adjacent vertebrae. FIGS. 1-2 illustrate the insertion of a pair of the Ray '373 fusion cages positioned within an intervertebral space.

### SUMMARY OF THE INVENTION

Accordingly, the present disclosure is directed to further improvements in spinal fusion technology. In accordance with one preferred embodiment, an apparatus for spinal fusion includes a plurality of intersecting side walls arranged with respect to x, y and z axes of the implant member to define an enclosed structure having a first dimension along the z-axis greater than a second dimension along the x-axis. The implant member is insertable between the adjacent vertebrae in a direction generally parallel to the y-axis thereof and with the x-axis extending in the general direction of the axis of the spine such that the second smaller dimension at least partially spans the intervertebral space defined between the adjacent vertebrae. The implant member is secured within the adjacent vertebrae by rotation thereof about the y-axis whereby the first larger dimension spans the intervertebral space. An anchoring element positioned on a peripheral portion of the implant member engages a respective vertebral portion to secure the implant member therein. Several of the walls have apertures extending to an internal cavity of the implant member to facilitate the fusion process. The implant member may include tool mating structure to facilitate insertion and manipulation of the implant member within the intervertebral space. Other embodiments and methods for spinal fusion are also disclosed.

### BRIEF DESCRIPTION OF THE DRAWINGS

Preferred embodiment(s) of the present disclosure are described herein with reference to the drawings wherein:

FIG. 1 is a view illustrating a portion of the vertebral column of a patient;

FIG. 2 is a view taken along line 2-2 of FIG. 1 illustrating a pair of prior art fusion implants positioned within the intervertebral space for fusion of adjacent vertebrae;

FIG. 3 is a perspective view of the fusion implant apparatus in accordance with the principles of the present disclosure;

FIG. 4 is an axial view of the implant apparatus;

FIG. 5 is a side plan view of the implant apparatus;

FIG. 6 is a partial perspective view of an insertion instrument contemplated for use in positioning the implant apparatus within adjacent vertebrae;

FIGS. 7-8 are views illustrating a preferred method of insertion of the implant apparatus; and

FIG. 9 illustrates a pair of the implant positioned within the intervertebral space.

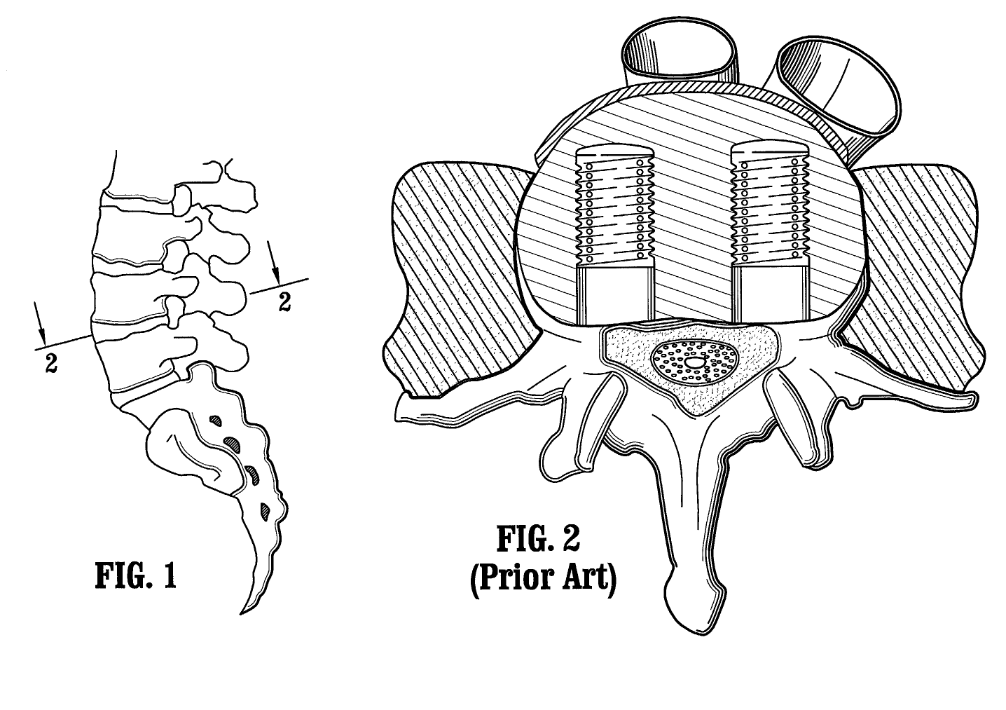

### DETAILED DESCRIPTION OF PREFERRED EMBODIMENTS

The preferred embodiment of the apparatus and method disclosed herein are discussed in terms of orthopedic spinal fusion procedures and instrumentation. It is envisioned, however, that the disclosure is applicable to a wide variety of procedures including, but, not limited to ligament repair, joint repair or replacement, non-union fractures, facial reconstruction and spinal stabilization. In addition, it is believed that the present method and apparatus finds application in both open and minimally invasive procedures including endoscopic and arthroscopic procedures wherein access to the surgical site is achieved through a cannula or small incision.

The following discussion includes a description of the fusion implant utilized in performing a spinal fusion followed by a description of the preferred method for spinal fusion in accordance with the present disclosure.

In the discussion which follows, the term “proximal”, as is traditional, will refer to the portion of the structure which is closer to the operator while the term “distal” will refer to the portion which is further from the operator.

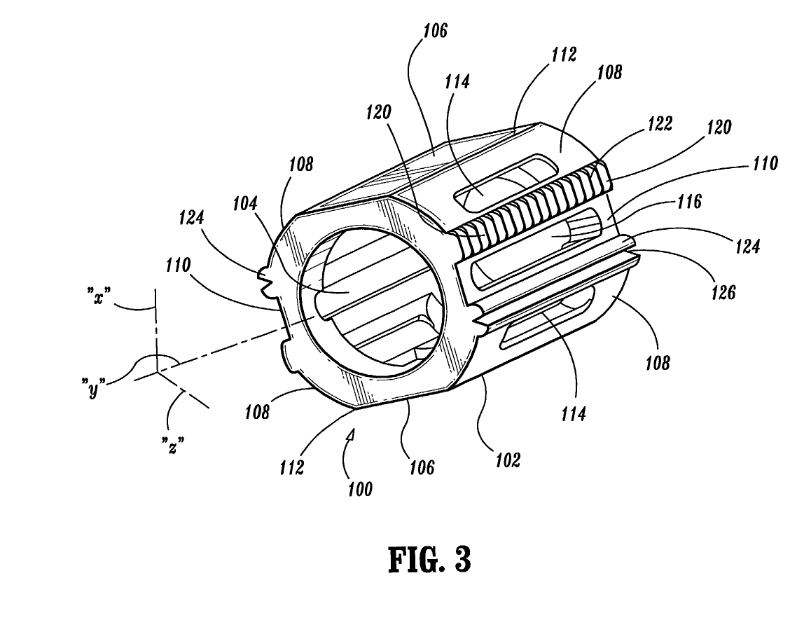

Referring now to the drawings in which like reference numerals identify similar or identical elements throughout the several views, FIG. 3 illustrates, in perspective, the fusion implant of the present disclosure. Fusion implant 100 is intended to be inserted within a preformed bore in adjacent bone structures, e.g., adjacent vertebrae, without necessitating tapping of an internal thread within the bone structures prior to insertion. Fusion implant 100 includes elongated implant body 102 which is preferably fabricated from a suitable biocompatible rigid material such as titanium and/or alloys of titanium, stainless steel, ceramic materials or rigid polymeric materials. Implant body 102 is preferably sufficient in strength to at least partially replace the supporting function of an intervertebral disc, i.e., to maintain adjacent vertebrae in desired spaced relation, during healing and fusion.

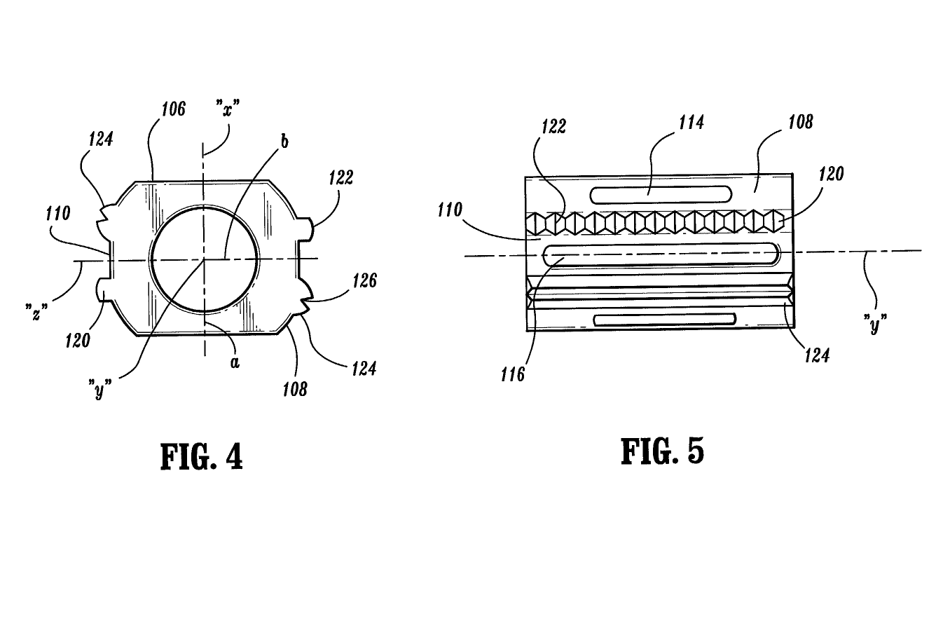

With reference now to FIG. 3, in conjunction with FIGS. 4-5 implant body 102 defines an x, y, z axes coordinate system with the y axis being the longitudinal axis of the implant body 102. Implant body 102 possesses a plurality of intersecting side walls, e.g., primary, secondary and tertiary side walls 106, 108, 110, respectively, which are connected to each other and arranged about the x, y and z axes to define the enclosed structure shown. Implant body 102 defines an inner cavity 104 within the interior of the implant body 102. Inner cavity 104 accommodates bone growth inducing substances such as bone chips, morphogenic drugs, etc. which facilitate the fusion process.

Primary side walls 106 are substantially planar and extend along the length of implant body 102. Primary side walls 106 are arranged in general parallel diametrically opposed relation to each other at opposite ends of the “x” axis and facilitate insertion and retention of the implant body 102 within the intervertebral space as will be discussed. Primary side walls 106 are devoid of apertures. This arrangement minimizes the potential for lateral growth of vertebral bone tissue within implant 100 after insertion.

Secondary side walls 108 are disposed adjacent primary side walls 106 and intersect the respective primary side walls 106 at an oblique angle along longitudinal edges 112. Secondary side walls 108 are generally arcuate although it is envisioned that the secondary side walls could be planar as well. Secondary side walls 108 each include at least one elongated aperture 114 extending through the side wall 108 in communication with inner cavity 104. Apertures 114 permit bone growth through implant body 102 and fusion with the bone growth inducing substance therein.

Tertiary side walls 110 are disposed between adjacent secondary side walls 108. Tertiary side walls 110 are also substantially planar and include an elongated aperture 116 extending through to communicate with inner cavity 104. Elongated aperture 116 is preferably substantially greater in dimension, i.e., longer than aperture 114 of secondary side walls 108. The aforedescribed configuration of implant body 102 provides a first cross-sectional dimension “a” along the x-axis which is substantially less than the second cross-sectional dimension “b” along the z-axis as best depicted in FIG. 4. Such configuration provides significant advantages in placement and securement of implant 100 as will be discussed.

Implant body 102 further includes a pair of longitudinal rows of fixation or anchoring members 120 adjacent the intersection of secondary and tertiary side walls 108, 110. Fixation members 120 are advantageously dimensioned to engage the adjacent vertebral bone structure to permanently fix the apparatus within the adjacent vertebrae. Fixation members 120 may include sharpened pointed crests 122 to facilitate penetration within the intervertebral beds. The longitudinal rows of fixation members 120 are disposed in diametrical opposed relation which thereby permits rotation of the implant body 102 in either direction within the intervertebral space to lock the implant therein.

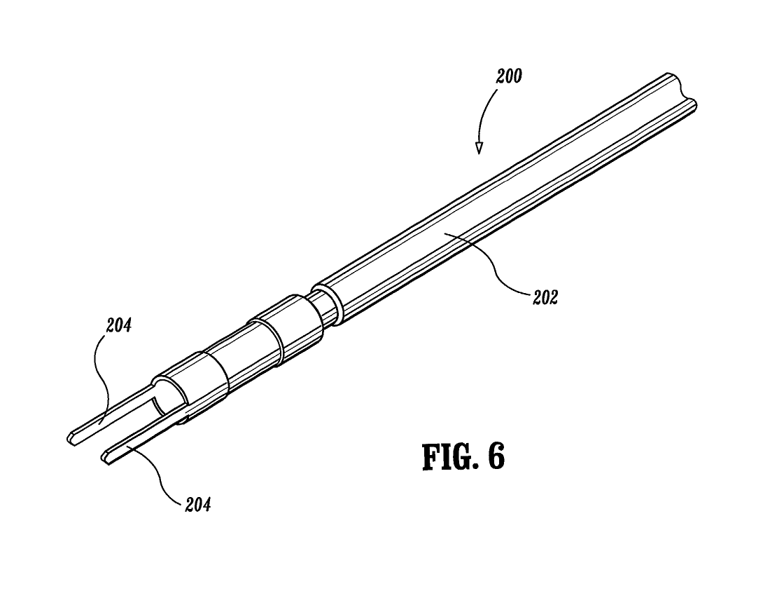

Implant body 102 further includes tool receiving structure in the form of longitudinal extending projections 124 extending the length of the implant body 102 in diametrically opposed relation. Each longitudinal projection 124 defines a tool receiving recess 126 to receive an insertion tool which facilitates insertion of the implant 100 with respect to the intervertebral space. One example of an insertion tool is disclosed in FIG. 6. This insertion tool 200 includes an elongated member 202 and a pair of longitudinally extending prongs 204 at the distal end of the elongated member 202. Prongs 204 are dimensioned to be received within corresponding recesses 126 of the longitudinal projections 124 whereby rotational movement of the insertion tool via a handle (not shown) connected to the elongated member 202 causes corresponding rotation of implant body 102. Other insertion tools and tool engaging structure are envisioned including the system disclosed in commonly assigned U.S. Pat. No. 5,885,294 to Winslow, the contents of which are incorporated herein by reference. It is also envisioned that projections 124 may be replaced with additional rows of fixation members 120 to provide additional securement.

The insertion of the fusion implant 100 into an intervertebral space defined between adjacent lumbar vertebrae will now be described. The subsequent description will be particularly discussed in conjunction with a posterior approach for spinal fusion implant insertion, however, it is to be appreciated that other approaches, e.g., direct anterior, lateral, posterior lateral, etc., are contemplated as well. Laparoscopic approaches are also envisioned.

The intervertebral space is accessed utilizing appropriate retractors to expose the posterior vertebral surface. A drilling instrument is utilized to prepare the disc space and vertebral end plates for insertion of the fusion implant. The cutting depth of drilling instrument may be adjusted as desired. The drilling instrument is advanced into the intervertebral space to shear the soft tissue and cut the bone of the adjacent vertebrae thereby forming a bore which extends into the adjacent vertebrae.

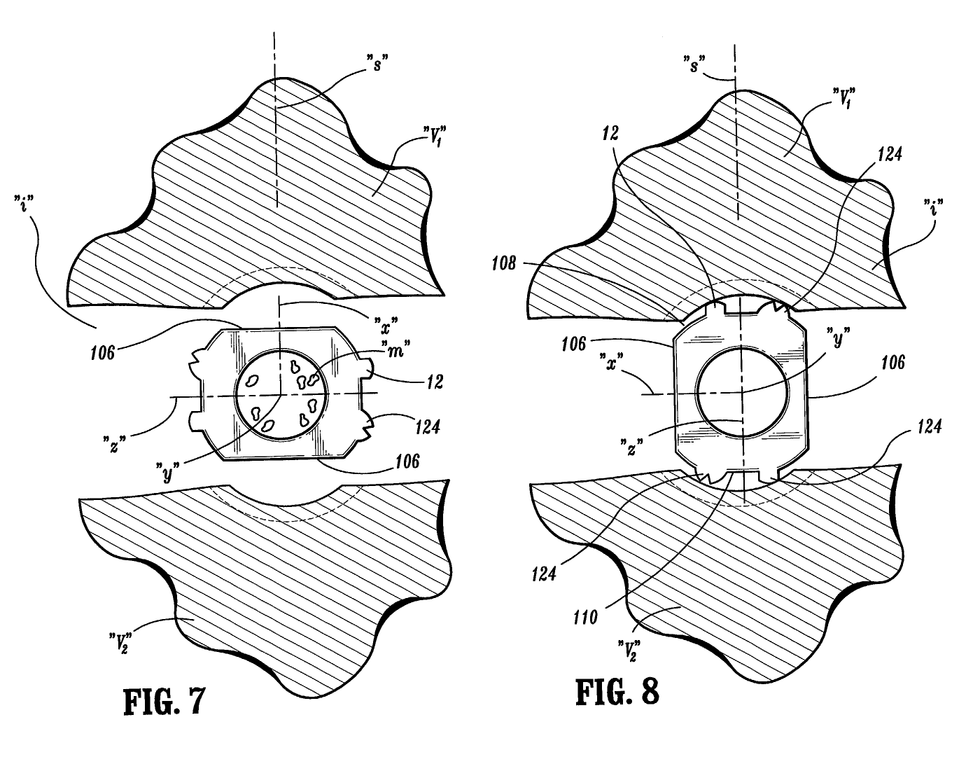

With reference now to FIG. 7, fusion implant 100 is packed with bone growth inducing substances “m” as is conventional in the art. The fusion implant 100 may then be mounted on insertion instrument 200 by positioning distal prongs 204 of insertion instrument 200 within correspondingly dimensioned recesses 126. For ease of illustration, however, in the drawings, the insertion instrument 200 is not shown. Implant 100 is arranged such that its x-axis extends in the general direction of the axis “s” of the spine and the z-axis is transverse to the spine axis “s”or parallel to the vertebral end plates of adjacent vertebrae “V₁, V₂”. Such arrangement presents the smaller cross-sectional x-dimension and planar surfaces 106 to the intervertebral space “i” thereby facilitating initial positioning between the adjacent vertebrae “V₁, V₂”. Implant member 100 is then advanced in a general direction parallel to its y-axis and into the intervertebral space “i” as depicted in FIG. 7. It is envisioned that a retractor (not shown) suitable for spinal distraction may be utilized to distract or maintain the adjacent vertebrae “V₁, V₂” in the spaced relation shown in FIG. 7. Thereafter, insertion instrument 200 is rotated 90° in either direction to thereby cause corresponding rotation of fusion implant 100 about the y-axis to the position depicted in FIG. 8. In this position, fixation members 120 engage respective vertebral portions “V₁, V₂” to secure implant member 100 to the vertebrae.

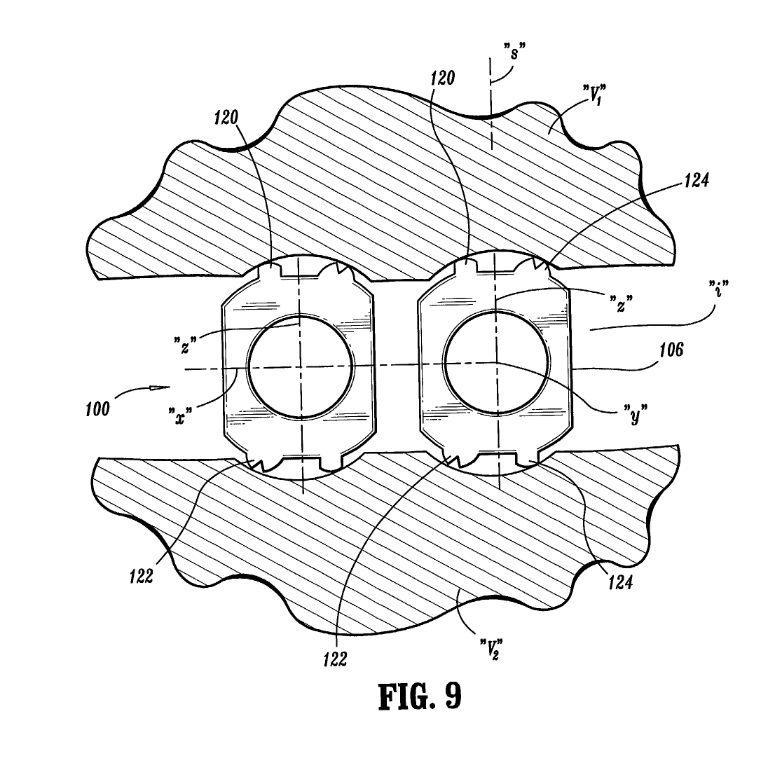

Thereafter, a second lateral side of the intervertebral space “i” is accessed and the above-described process is repeated to insert a second implant 100 in lateral side-by-side relation as shown in FIG. 9. As appreciated, implants 100 are arranged such that respective planar surfaces 106 of the implants 100 are disposed in adjacent side-by-side relation. Such arrangement permits implants 100 to be placed in closer proximity within the intervertebral space “i”.

Implants 100 form struts across the intervertebral space “i” to maintain the adjacent vertebrae “V₁, V₂” in appropriate spaced relation during the fusion process. Over a period of time, the adjacent vertebral tissue communicates through apertures 114, 116 of respective secondary and tertiary side walls 108, 110 within implants 100 to form a solid fusion. Desirably, lateral vertebral tissue growth into the implant 100 is restricted due to primary side wall 100 being devoid of apertures. Such lateral growth would inhibit the fusion process and potentially restrict subsequent spinal mobility.

From the foregoing and with reference to the various figure drawings, those skilled in the art will appreciate that certain modifications can also be made to the present disclosure without departing from the scope of the same. While the above description contains many specifics, these specifics should not be construed as limitations on the scope of the disclosure, but merely as exemplifications of preferred embodiments thereof. Those skilled in the art will envision other modifications within the scope and spirit of the claims appended hereto.

What is claimed is:

1. An apparatus for facilitating fusion of adjacent vertebrae of the spine, which comprises:
an implant member dimensioned for insertion within an intervertebral space between adjacent vertebrae to retain the vertebrae in spaced relation for a sufficient length of time to facilitate fusion and healing, the implant member including a plurality of intersecting side walls arranged about a longitudinal axis of the implant member to define an internal cavity for reception of bone growth inducing substances, at least two of the side walls having an aperture extending to communicate with the internal cavity to facilitate direct bone ingrowth therethrough, at least one of the side walls extending continuously along a longitudinal length of the implant member and being devoid of an aperture whereby upon insertion of the implant member, the one side wall extends in a general direction of an axis of the spine in non-contacting relation with respect to the adjacent vertebrae.

2. The apparatus according to claim 1 wherein the one side wall is substantially planar.

3. The apparatus according to claim 3 including a pair of the one side wall arranged in general opposed parallel relation.

4. The apparatus according to claim 1 further including a plurality of fixation segments arranged in general longitudinal alignment with respect to the longitudinal axis of the implant member.

5. The apparatus according to claim 3 wherein the fixation segments are generally disposed at the intersection of two adjacent side walls.

6. The apparatus according to claim 5 including first and second longitudinal rows of fixation segments.

7. The apparatus according to claim 6 wherein the first and second longitudinal rows are disposed in diametrically opposed relation with respect to the longitudinal axis.

8. The apparatus according to claim 4 wherein the implant member includes tool mating structure to facilitate insertion and manipulation of the implant member within the intervertebral space.

9. The apparatus according to claim 8 wherein the tool mating structure includes a tool receiving projection on the exterior of the implant member and extending at least a portion of the length of the implant member.

10. The apparatus according to claim 9 including at least two tool receiving projections on the exterior of the implant member.

11. The apparatus according to claim 1 wherein the implant member defines first and second cross-sectional dimensions transverse to the longitudinal axis, the first cross-sectional dimension being greater than the second-cross-sectional dimension.

12. An apparatus for facilitating fusion of adjacent vertebrae of the spine, which comprises:
an implant member dimensioned for insertion within an intervertebral space between adjacent vertebrae to retain the vertebrae in spaced relation during healing, the implant member including a plurality of intersecting side walls arranged with respect to x, y and z axes of the implant member, the implant member having a first dimension along the z-axis greater than a second dimension along the x-axis, the implant member being insertable between the adjacent vertebrae in a direction generally parallel to the y-axis thereof and with the x-axis extending in the general direction of the axis of the spine such that the second smaller dimension at least partially spans the intervertebral space defined between the adjacent vertebrae, the implant member being secured within the adjacent vertebrae by rotation thereof about the y-axis whereby the first larger dimension spans the intervertebral space and an anchoring element positioned on a peripheral portion of the implant member engages a respective vertebral portion to secure the implant member therein.

13. The apparatus according to claim 12 wherein the implant member includes a pair of parallel planar side walls arranged in opposed relation and intersected by the x-axis.

14. A method for fusing adjacent vertebrae, comprising the steps of:
accessing an intervertebral space defined between upper and lower adjacent vertebral portions;
providing an implant apparatus including an implant member having a plurality of intersecting side walls, and defining a longitudinal axis, at least one of the side walls being substantially planar, at least two of the side walls having contacting surfaces which facilitate bone ingrowth, the implant member including fixation members;
advancing the implant apparatus into the intervertebral space such that the one side wall is in parallel relation with one of the upper and lower adjacent vertebral portions;
rotating the implant member about the longitudinal axis to cause the fixation members to engage at least one of the upper and lower vertebral portions thereby securing the implant apparatus within the intervertebral space; and
permitting bone ingrowth into the contacting surfaces of the at least two side walls of the implant apparatus.

15. The method according to claim 14 wherein the implant member defines an internal cavity and further including the step of introducing bone growth inducing substances within the internal cavity.

16. The method according to claim 15 wherein the at least two side walls include apertures in communication with the internal cavity and wherein, during the step of permitting, vertebral bone tissue communicates with the bone growth inducing substances.

17. The method according to claim 16 wherein the implant member includes first and second longitudinal rows of fixation segments and wherein during the step of rotating the first and second longitudinal rows respectively engage upper and lower adjacent vertebrae portions.

18. The method according to claim 17 wherein the implant member includes first and second substantially planar side walls arranged in diametrical opposed relation and wherein the step of advancing includes positioning each side wall to be in parallel relation with respective upper and lower vertebral portions.
